(19) United States
(12) Patent Application Publication
(10) Pub. No.: US 2020/0022630 A1
(43) Pub. Date: Jan. 23, 2020

(54) REMOVAL PORT FOR REMOVING A FLUID, SYSTEM AND USE OF A REMOVAL PORT

(71) Applicant: Hans HAINDL, Wennigsen (DE)

(72) Inventor: Hans HAINDL, Wennigsen (DE)

(21) Appl. No.: 16/097,085

(22) PCT Filed: Apr. 25, 2017
(86) PCT No.: PCT/EP2017/059755

§ 371 Date: May 3, 2019

(30) Foreign Application Priority Data
Apr. 27, 2016 (DE) 102016005072.1

(51) Int. Cl. A61B 5/15

(52) U.S. Cl. CPC A61B 5/150992

(57) ABSTRACT

A withdrawal port (10) for withdrawing a liquid, in particular blood, from a tubing comprises a first tube connection (12), a second tube connection (13), and a fluid connection (15) between the first and second tube connections (12, 13). The withdrawal port (10) comprises a receiving section (17) suitable for reversibly releasably receiving a syringe cone (42) of a syringe (41). The withdrawal port (10) is configured such that an insertion of the syringe cone (42) into the receiving section (17) interrupts the fluid connection (15) between the first tube connection (12) and the second tube connection (13) and causes fluid communication of the syringe cone (42) with the first tube connection (12).

The present invention relates to a withdrawal port for withdrawing a liquid, in particular blood, from a tubing.

In different medical treatments, e.g., in cardiac catheterization or in intensive care, it may be required to frequently withdraw blood from a patient. This may be necessary, e.g., in order to analyze the withdrawn blood. In such treatments, the patient often has an arterial or venous cannula inserted into a blood vessel. In order not to have to puncture the patient anew every time when blood is withdrawn, it is desirable to withdraw the blood from the tubing for analytical purposes.

For this purpose, the prior art discloses, e.g., a three-way stopcock by means of which an infusion line is shut off towards an infusion bottle, and then first an infusion solution and later blood can be withdrawn from the arm of the three-way stopcock. A problem occurring with conventional three-way stopcocks is that blood diluted with an infusion solution must be withdrawn first in order to avoid distorted laboratory results. Therefore, first a portion of said mixed blood must be withdrawn with a syringe. As soon as undiluted blood is suspected, the syringe is replaced, and the undiluted blood is withdrawn and analyzed. The mixed blood that has been withdrawn is discarded. The result is that always more blood has to be withdrawn from the patient than is actually necessary for the analysis. Under certain circumstances, this may lead to anemia in the course of treatment. Furthermore, such techniques are critical also from a hygienic point of view since blood residues, in which germs may multiply, may remain in the three-way stopcock's arm used for blood withdrawal.

In order to reduce or completely eliminate such elevated blood losses, different systems may be deployed in which the withdrawn blood is returned to the patient in a closed system. These systems involve, e.g., a system as described in EP 1 017 317 A1.

Such systems consist of a liquid reservoir which is arranged within a conduit leading to the vascular cannula. The infusion solution flows to the patient through said liquid reservoir. A liquid may be withdrawn from the tubing, e.g., by pulling a plunger in a cylinder, in order for it to be filled into said reservoir. In doing so, just as much liquid is withdrawn from the tubing until the arm of the tubing from the reservoir to the patient is completely filled with undiluted blood. On said arm of the tubing, there is a withdrawal port which can be pierced, e.g., with a particular cannula or directly with the syringe cone, and from which blood can be withdrawn, wherein the port closes automatically after blood withdrawal. After this process, the reservoir is drained again, e.g., by advancing the plunger, and the mixed blood is returned to the patient. An advantage of this arrangement is that the patient regains the unneeded blood, and that typically there are no significant blood residues that remain in a dead space, as is the case with a three-way stopcock, which thus poses a risk of infection. Usually, the withdrawal ports for blood withdrawal can be disinfected without problems.

In case such a withdrawal port is arranged in the tubing for blood withdrawal, it must be guaranteed that the blood is withdrawn by said withdrawal port actually at the side facing the patient, i.e., the blood is undiluted, and that no blood from the side facing away from the patient, i.e., mixed blood, is withdrawn from the reservoir. For this purpose, a stopcock is often arranged at the side of the withdrawal port facing away from the patient. In some withdrawal ports, such a stopcock is also incorporated into the housing of the withdrawal port. Such a withdrawal port is described in, e.g., EP 1 234 596 A1.

Such stopcocks must be operated by medical staff. However, the risk of incorrect operation still exists. For instance, there may be times when they forget to close the stopcock or open it again after blood withdrawal. In the former case, the blood may be drawn in from the side facing away from the patient, which may lead to incorrect analysis results. Withdrawal ports with integrated cocks pose a reduced risk of incorrect operation since without shifting the cock no blood can be withdrawn, but they still entail disadvantages. For instance, syringe cones comprise considerable tolerances in length and diameter. Since the cock, however, is arranged quite closely below a membrane of the port as a rigid component, it may happen that particular syringe cones impact on the cock and are thus not properly clamped in the guide of the withdrawal port, so that accidental detachment of the syringe from the withdrawal port (a so-called “disconnection”) may occur during blood withdrawal.

It is thus an object of the present invention to provide an improved withdrawal port for blood withdrawal which takes into consideration the aforementioned problems.

This and further objects are achieved with a withdrawal port, a system, and a use according to the independent claims. Further preferred embodiments are described in the independent claims.

A withdrawal port according to the invention is suitable for withdrawing a liquid, in particular blood, from a tubing. The withdrawal port comprises a first tube connection and a second tube connection. The first tube connection may be connected or connectable with an arm of a tubing facing the patient. The second tube connection may, e.g., be connected or connectable with an arm facing away from the patient and leading to a liquid reservoir, wherein other uses of the withdrawal port according to the invention are also possible.

The withdrawal port comprises a fluid connection, in particular a liquid connection, between the first tube connection and the second tube connection. The withdrawal port may comprise a receiving section. The receiving section may be suitable for reversibly releasably receiving a syringe section of a syringe. The syringe section may in particular be a syringe cone. The withdrawal port is configured such that an insertion of the syringe section into the receiving section interrupts the fluid connection between the first tube connection and the second tube connection. The withdrawal port may be configured such that the insertion of the syringe section into the receiving section causes fluid communication, in particular liquid communication, of the syringe section, e.g., the syringe cone, with the first tube connection.

In such a withdrawal port, the insertion of the syringe cone into the receiving section of the withdrawal port automatically causes an interruption of the fluid connection between the first tube connection and the second tube connection. The fluid connection may in particular be a liquid connection. By interrupting the liquid connection, a flow of liquid between the first tube connection and the second tube connection is substantially disabled. The risk of accidentally drawing in mixed blood from a reservoir containing mixed blood is reduced. In an ideal situation, there is no risk of accidentally drawing in a liquid from another arm than the first arm of the tubing. Due to the reduced risk of handling errors, the risk of distorted analysis results is reduced when further analyzing the blood.

The interruption of the fluid connection between the first tube connection and the second tube connection does not necessarily require the second tube connection to be completely fluidly separated from the first tube connection. It is sufficient that a flow resistance for a liquid flow between the first and second tube connections is larger, preferably much larger, than a flow resistance between the first tube connection and the syringe cone when the latter is inserted into the withdrawal port. However, it is preferred that the liquid flow (i.e., in particular the blood flow) between the first and second tube connections is substantially and, particularly preferred, completely interrupted and/or disabled.

Preferably, the withdrawal port comprises a housing and a closure section. The closure section may be configured to reversibly releasably engage the syringe section in order to interrupt the fluid connection between the first tube connection and the second tube connection. In doing so, the interruption of the fluid connection may, e.g., be achieved by way of deforming the closure section under the influence of the syringe cone, and/or by way of cooperation of the syringe cone and the closure section in another way so as to interrupt the fluid connection between the first and the second tube connection while the syringe cone is in liquid communication with the first tube connection.

The closure section may comprise a sealing surface on the housing of the withdrawal port. The sealing surface may be arranged such that the second tube connection is closed, e.g., in a substantially fluid-tight manner, when the syringe section comes into contact with the sealing surface. The sealing engagement between the syringe section and the housing may be guaranteed, e.g., by tapering at least a part of an inner wall of the housing to such an extent that the inserted syringe cone contacts the inner wall safely along a continuous, e.g., substantially annular, contact line.

The withdrawal port may be configured such that the first tube connection comprises a first longitudinal axis, and the second tube connection comprises a second longitudinal axis offset from the first longitudinal axis. In a withdrawal port with a simple geometry, such an arrangement of the first and second tube connections allows the fluid connection between the first tube connection and the second tube connection to be interrupted by means of the syringe cone's contact with the sealing surface. The first longitudinal axis of the first tube connection and the second longitudinal axis of the second tube connection may run parallel to each other. The first longitudinal axis of the first tube connection and the second longitudinal axis of the second tube connection may preferably be offset from each other along an insertion direction along which the syringe cone is insertable into the withdrawal port.

The housing may be configured to redirect a liquid flow between the first tube connection and the second tube connection when the syringe section is not inserted into the receiving section. For this purpose, the housing may comprise at least one wall running transversely, in particular substantially perpendicular, to the first longitudinal axis of the first tube connection.

The closure section may preferably be elastically deformable in order to close, in particular in a substantially fluid-tight manner, the second tube connection when inserting the syringe section into the receiving section. The closure section may comprise a region, e.g., a substantially frustoconical region, running around a longitudinal axis of the second tube connection, said region being substantially sealingly compressed by the syringe section during insertion of the syringe section.

In order to ensure that the syringe section is still in good fluid communication with the first tube connection even when contacting the elastically deformable closure section, the housing may define an insertion direction for the syringe cone such that the insertion direction is inclined both to a first longitudinal axis of the first tube connection and to a second longitudinal axis of the second tube connection.

Preferably, the housing may define the insertion direction for the syringe cone such that the insertion direction forms an obtuse angle with the first longitudinal axis of the first tube connection. The housing may determine the insertion direction for the syringe cone such that the insertion direction forms an acute angle with the second longitudinal axis of the second tube connection. The angle may be greater than 60°, e.g., from 60 to 90°.

The elastically deformable closure section may be reversibly converted from a first configuration into a second configuration by means of inserting the syringe section into the receiving section. In the first configuration, the closure section may be arranged in the housing of the withdrawal port such that the closure section leaves open both the first tube connection and the second tube connection in order to enable a liquid connection between the two. In the second configuration, the elastically deformable closure section may close the second tube connection while preferably a fluid communication between the first tube connection and the syringe cone is established.

The housing may comprise an aperture through which the syringe section is insertable into the receiving section of the withdrawal port. The elastically deformable closure section may be configured to close and/or seal the aperture in the first configuration. In doing so, the risk of contamination of the withdrawal port may easily be prevented even without additional components.

The elastically deformable closure section may consist of an elastomeric material, or may comprise an elastomeric material. The elastomeric material may, e.g., comprise silicone, latex, a thermoplastic elastomer (TPE), and/or ethylene propylene diene rubber (EPDM).

The closure section may be configured so as to frictionally retain the syringe section inserted into the receiving section within the housing alone or in conjunction with the housing, in particular in conjunction with a female conical section. Alternatively, the syringe section may also be retained by the housing alone. The closure section may be configured to retain in a clamped manner the syringe section inserted into the receiving section. Thus, the risk that the syringe accidentally detaches from the withdrawal port is reduced.

Preferably, the housing of the withdrawal port comprises an aperture through which the syringe section is insertable into the receiving section of the withdrawal port. The withdrawal port may comprise a seal arranged at the aperture. The seal may be elastically deformable for sealing engagement with syringe cones of different diameters. Alternatively or additionally, the housing may be elastically deformable at the aperture for sealing engagement with syringe cones of different diameters.

The withdrawal port may be configured so as to frictionally retain the syringe section at the withdrawal port, in particular with a radially-inwardly directed clamping force, when the syringe section is inserted into the receiving section.

The withdrawal port may comprise a retainer for retaining the seal. The retainer may be configured as a cap which partially engages over the seal. The retainer may be configured so as to frictionally retain the syringe section on the withdrawal port, in particular with a radially-inwardly directed clamping force, when the syringe section is inserted into the receiving section. Alternatively or additionally, the seal may be configured so as to frictionally retain the syringe section on the withdrawal port, in particular with a radially-inwardly directed clamping force, when the syringe section is inserted into the receiving section.

The seal may consist of an elastomer, e.g., silicone, latex, TPE, and/or EPDM.

The seal may comprise a protrusion, e.g., a nose, for sealingly abutting a surface of the housing. The nose may be provided such that it extends from an inner surface of the housing into the aperture. An outer surface of the nose may be substantially flush with an outer surface of the housing when the syringe cone is not inserted into the receiving section.

The protrusion of the seal extending into the aperture may form a transition without any steps between the outer surface of the seal and the surrounding outer surface of the housing and/or the surrounding outer surface of the retainer retaining the seal. In the vicinity of the aperture, an outer surface of the withdrawal port without steps may thus be provided for wipe disinfection when the syringe section is not inserted into the withdrawal port. A well-disinfectable outer surface of the withdrawal port without steps is formed.

The housing and/or the seal may be configured to frictionally retain the syringe section at a section of the syringe cone spaced apart from an end of the syringe cone. The housing and/or the seal may be configured to clamp and/or retain in a clamped manner the syringe section at a section of the syringe cone spaced apart from an end of the syringe cone. The risk of the syringe accidentally detaching from the withdrawal port is thus reduced.

The withdrawal port may be configured to retain the syringe section in a clamped manner without the syringe section being threadingly engaged with the withdrawal port and/or otherwise coupled in a form-fit manner.

The withdrawal port may be configured to frictionally retain the syringe section within the housing at at least one end region of the syringe section. The withdrawal port may be configured to clamp and/or retain in a clamped manner the syringe section within the housing at at least an end region of the syringe section. The risk of the syringe accidentally detaching from the withdrawal port is thus reduced.

The housing may comprise protrusions, in particular ridges, for guiding or frictionally retaining the syringe section. The protrusions may be provided at the aperture, e.g., at at least two positions circumferentially around the aperture in order to guide the syringe cone. The protrusions may define an insertion direction in which the syringe cone can be inserted into the withdrawal port.

The withdrawal port may comprise at least one retaining plate for retaining and/or fastening the withdrawal port. The retaining plate may be suitable for fastening the withdrawal port to a patient's skin or to a different structure, e.g., a structure of a bed, an operating table, or an infusion pole. The retaining plate may be dimensioned such that the withdrawal port may be retained with two fingers at the retaining plate while the syringe cone is being inserted.

The withdrawal port according to any one of the disclosed embodiments may be configured to allow for blood withdrawal without substantially limiting the flow cross-section. The withdrawal port may be configured such that the flow cross-section between the first tube connection and the receiving section is not reduced when the syringe section is being inserted into the receiving section. The withdrawal port may alternatively or additionally be configured such that the withdrawal port does not overlap the syringe aperture when the syringe section is completely inserted into the receiving section. In particular, the withdrawal port may be configured such that the seal of the withdrawal port abuts an outer surface of the syringe cone spaced apart from the syringe opening when the syringe section is completely inserted into the receiving section.

The withdrawal port according to any one of the disclosed embodiments may be configured such that the withdrawal port flushes itself after removal of the syringe section from the withdrawal port. For this purpose, the withdrawal port may comprise a nose at the inlet facing away from the patient.

A system according to the invention comprises the withdrawal port according to the invention as well as a syringe. The syringe comprises a syringe cone suitable for being reversibly releasably inserted into the receiving section of the withdrawal port in order to withdraw blood from a tubing by means of the withdrawal port.

In such a system, the insertion of the syringe cone into the receiving section of the withdrawal port automatically causes the fluid connection between the first and second tube connections to be interrupted. The risk of accidentally drawing in mixed blood from a reservoir containing mixed blood is thus reduced. Furthermore, when further analyzing the blood, the risk of distorted analysis results is reduced due to the reduced risk of handling errors.

The system may be configured such that by means of inserting the syringe cone into the receiving section the system can be converted between a first configuration, in which the syringe cone is arranged outside of the receiving section and the fluid connection between the first tube connection and the second tube connection is open, and a second configuration.

In the second configuration of the system, the syringe cone is inserted into the receiving section of the withdrawal port as far as it will go, the fluid connection between the first tube connection and the second tube connection is interrupted, and the syringe cone is in fluid communication with the first tube connection.

The system may comprise a first arm of a tubing that is connected to the first tube connection. The system may comprise a second arm of a tubing that is connected to the second tube connection. The second arm of the tubing may be connected to a liquid reservoir which may, e.g., contain a mixture of blood and an infusion solution.

In a method according to the invention, the withdrawal port according to the invention is used for withdrawing blood from a tubing.

In the withdrawal port according to the invention, the system according to the invention, and the use according to the invention, the fluid connection between the first and second tube connections may in each case be a liquid connection. Said liquid connection may be interrupted so that substantially no liquid flow between the first and second tube connections is possible when the syringe cone is inserted into the withdrawal port as far as it will go. The simultaneously enabled fluid communication between the syringe cone and the first tube connection may be a liquid communication allowing a liquid to be drawn in through the first tube connection into the syringe cone.

In the following, preferred embodiments of the invention are described in detail with reference to the figures, in which:

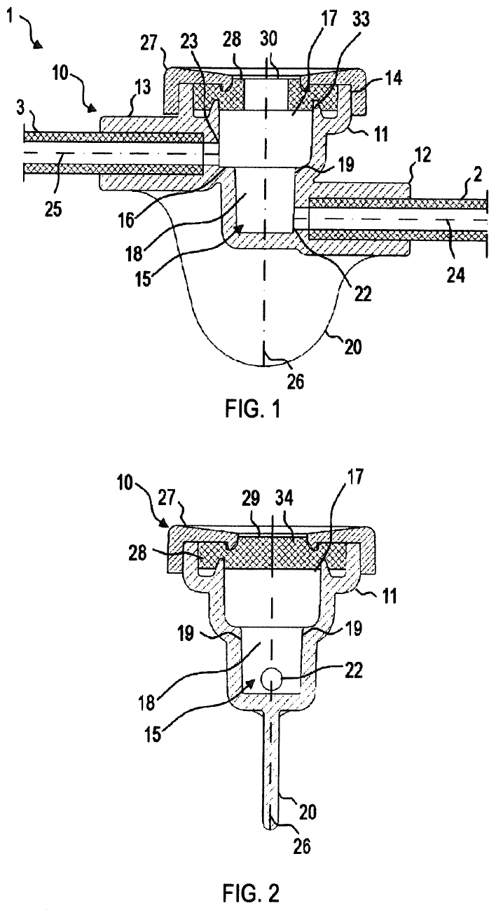

FIG. 1 shows a sectional view of a withdrawal port according to the invention when a fluid connection between a first and second tube connection is open;

FIG. 2 shows a sectional view of the withdrawal port according to FIG. 1 when the fluid connection between the first and second tube connection is open;

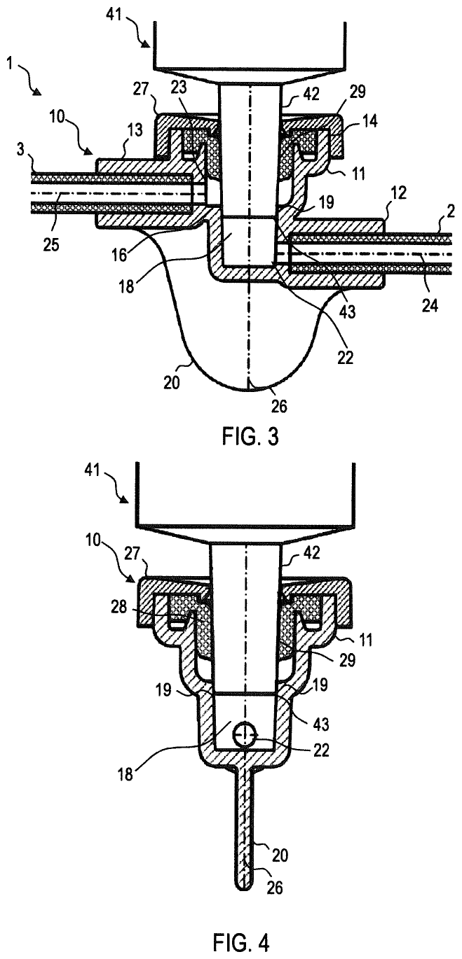

FIG. 3 shows a sectional view of the withdrawal port according to FIG. 1 when the fluid connection between the first and second tube connection is interrupted;

FIG. 4 shows a further sectional view of the withdrawal port according to FIG. 1 when the fluid connection between the first and second tube connection is interrupted;

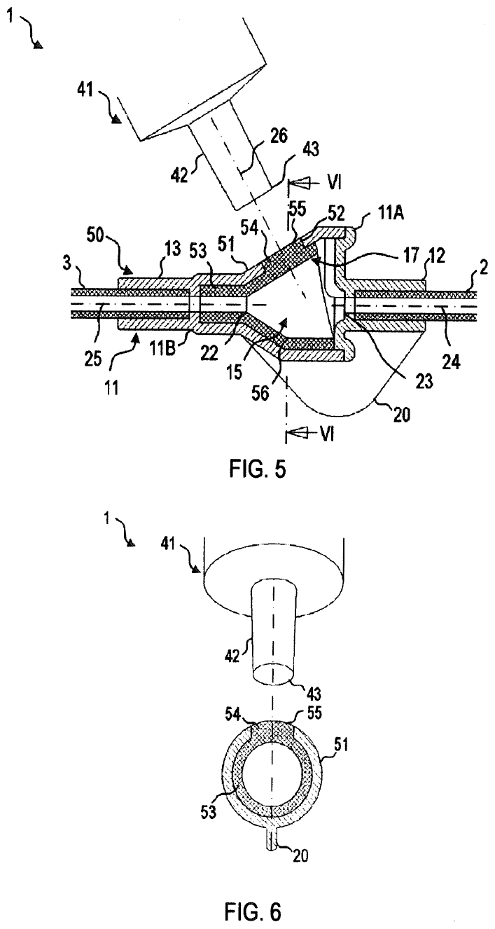

FIG. 5 shows a sectional view of a withdrawal port according to the invention when a fluid connection between a first and second tube connection is open;

FIG. 6 shows a further sectional view of the withdrawal port according to FIG. 5 along a sectional line VI-VI depicted in FIG. 5 when the fluid connection between the first and second tube connection is open;

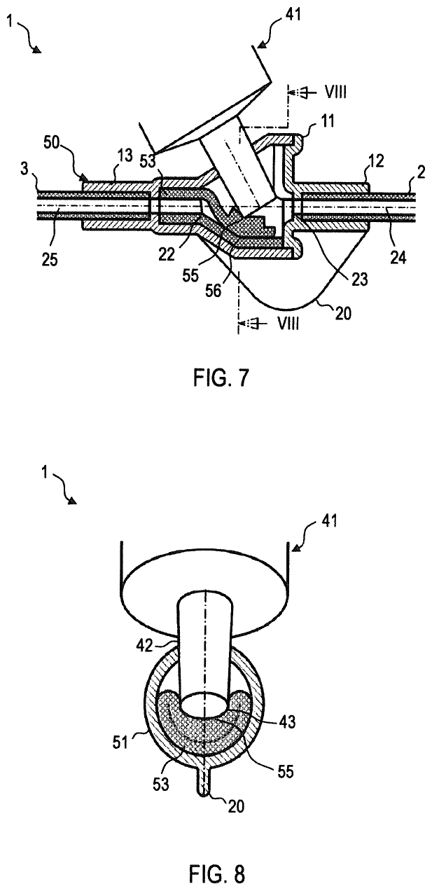

FIG. 7 shows a sectional view of the withdrawal port according to FIG. 5 when the fluid connection between the first and second tube connection is interrupted;

FIG. 8 shows a further sectional view of the withdrawal port according to FIG. 5 along the sectional line VIII-VIII depicted in FIG. 7 when the fluid connection between the first and second tube connection is interrupted;

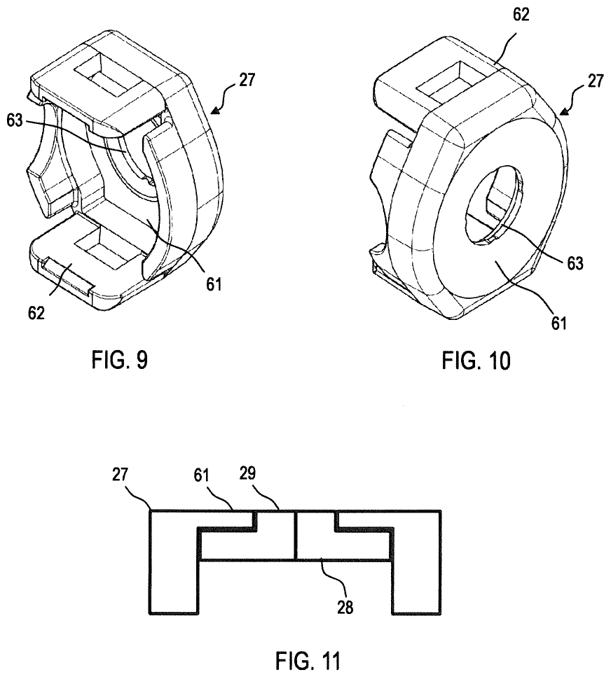

FIG. 9 shows a perspective view of a retainer of a seal of the withdrawal port according to FIG. 1;

FIG. 10 shows a further perspective view of a retainer of a seal of the withdrawal port according to FIG. 1; and

FIG. 11 shows a partially sectional view of the withdrawal port according to FIG. 1.

In the following, preferred and advantageous exemplary embodiments of the invention are described with reference to the figures, with identical reference signs designating identical or similar elements. The features of the different exemplary embodiments may be combined with each other unless this is explicitly ruled out in the following description.

FIGS. 1 to 4 show a system 1 with a withdrawal port 10 according to an embodiment. The withdrawal port 10 allows blood or another liquid to be withdrawn from a first arm 2 of a tubing. FIG. 1 shows a sectional view of the withdrawal port 10, wherein the section plane runs parallel to longitudinal axes 24, 25 of tube connections 12, 13, and the system 1 is depicted in a state in which the fluid connection is open. The fluid connection may particularly be a liquid connection 15. FIG. 2 shows a sectional view of the withdrawal port, wherein the section plane runs perpendicular to the longitudinal axes 24, 25 of the tube connections 12, 13, and the system 1 is depicted in a state in which the liquid connection 15 is open. FIG. 3 and FIG. 4 show the withdrawal port in a view corresponding to the sectional views of FIGS. 1 and 2 when the syringe cone 42 is inserted into the withdrawal port 10 and the liquid connection 15 is interrupted.

The withdrawal port 10 comprises a housing 11. The withdrawal port 10 comprises a first tube connection 12 and a second tube connection 13. When using the withdrawal port 10, the first tube connection 12 may serve as a connection with the arm 3 of the tubing facing the patient. The second tube connection 13 may, e.g., serve as a connection to a reservoir containing a mixture of blood and an infusion solution.

Offset from one another, the first tube connection 12 and the second tube connection 13 open into a liquid connection 15 of the housing in the withdrawal port 10. The liquid connection 15 allows for a liquid flow between a first orifice 22 of the first tube connection 12 and a second orifice 23 of the second tube connection 13 when the syringe cone 42 is not inserted into the withdrawal port 10. The liquid connection 15 may substantially be defined by the inner wall of the housing 11. The housing 11 comprises an aperture 33 via which the syringe cone 42 may be inserted into a receiving section 17 within the housing 11. The aperture may be defined by a housing section 14 and may, e.g., be circular. As will be described in more detail, the liquid connection 15 is automatically interrupted when the syringe cone 42 is being inserted into the withdrawal port 10 as far as it will go.

The aperture 33 of the housing may be provided with a suitable seal 28. For instance, the seal 28 may be arranged at the aperture in order to seal the aperture in the manner of a cap. The seal 28 may comprise a slit 30 illustrated in the sectional views of FIG. 1 and FIG. 3, said slit being closed by the elastic properties of the seal 28 when the syringe cone 42 is not inserted into the withdrawal port 10. The seal 28 may be mounted to the housing 11 by means of snapping, ultrasonic welding, or by other means. For example, a retainer 27 may sealingly retain in place the seal 28. The seal 28 may consist of an elastomeric material and, e.g., be an elastomeric membrane. The seal 28 may comprise, e.g., silicone, latex, a thermoplastic elastomer (TPE), and/or an ethylene propylene diene rubber (EPDM). An outer surface 29 of the seal 28 forming a protrusion may abut an outer surface of the syringe cone 42 under bias when the syringe cone 42 is inserted into the receiving section 17. The seal 28 may abut the outer surface of the syringe cone 42 in a substantially liquid-tight manner when the syringe cone 42 is inserted into the receiving section 17 in order to prevent liquid, in particular blood, from escaping.

FIG. 9 to FIG. 11 illustrate a possible configuration of the seal 28 and the retainer 27 of the withdrawal port according to an exemplary embodiment. The retainer 27 may be configured as a cap comprising an annular section which partially engages over the seal 28. The retainer 27 may comprise an annular shoulder 61. The retainer 27 may comprise a mounting section 62 for mounting the retainer 27 to the housing 11, e.g., by means of a snap-in connection.

The aperture 63 for inserting the syringe cone 42 into the housing 11 may be surrounded by the annular shoulder 61. In the mounted state of the seal 28 and the retainer 27, a protrusion 34 of the seal 28, e.g., a nose, may extend into the aperture 63. The protrusion 34 of the seal 28 may be dimensioned such that it is flush with the region of the retainer 27 surrounding the aperture 63 when the syringe cone 42 is not inserted into the withdrawal port 10. The protrusion of the seal 28 may be dimensioned such that together with the retainer 27 it provides an outer surface that is substantially free of steps around and at the aperture 63. Thereby, wipe disinfection may be facilitated. The protrusion of the seal 28 may be dimensioned such that it completely fills the aperture 63 of the retainer 27 without a recess, and together with the retainer 27 it forms an outer surface of the withdrawal port that is free of steps when the syringe cone 42 is not inserted into the withdrawal port 10.

In further exemplary embodiments, no retainer separate from the housing 11 has to be provided. The seal 28 may comprise a protrusion 34 which extends into an aperture of the housing 11. The protrusion 34 of the seal 28 may be dimensioned such that it is flush with the region of the housing 11 surrounding the aperture when the syringe cone 42 is not inserted into the withdrawal port. The protrusion 34 of the seal 28 may be dimensioned such that together with the housing 11 it provides an outer surface that is substantially free of steps around the aperture of the housing 11. Thereby, wipe disinfection may be facilitated. The protrusion 34 of the seal 28 may be dimensioned such that it completely fills the aperture of the retainer without a recess and forms an outer surface of the withdrawal port that is free of steps when the syringe cone 42 is not inserted into the withdrawal port.

The retainer 27 may be configured to retain in a clamped manner the syringe cone 42 at a section of the syringe cone 42 spaced apart from the end region 43 of the syringe cone. The retainer 27 may particularly be configured to retain in a clamped manner the syringe cone 42 at the proximal end of the syringe cone 42. In this way, the retainer 27 may exert an inwardly-directed clamping force on the syringe cone 42. The clamping force may be directed substantially radially to the center axis of the syringe cone 42.

The retainer 27 may act similar to a clamping disk on a shaft. In use, a slight elastic spring compression of the retainer 27 may occur and lead to a deadlock when the syringe cone recedes.

Alternatively or additionally, the withdrawal port 10 may be configured such that the seal 28 retains in a clamped manner the syringe at a section of the syringe cone 42 spaced apart from the end section 43 of the syringe cone. In this way, the seal 28 may exert an inwardly-directed clamping force on the syringe cone 42. The clamping force may be directed to the center axis of the syringe cone 42 substantially radially.

The withdrawal port 10 may thus be configured to retain the syringe cone 42 in a clamped manner on the withdrawal port 10 when the syringe cone 42 is inserted into the withdrawal port 10. The withdrawal port 10 may be configured such that the syringe cone 42 is retained by the withdrawal port 10 without it having to be screwed to the withdrawal port. The withdrawal port 10 may have an outer surface that is free of threads. Disinfection of the outer surfaces is thus facilitated in the vicinity of the aperture via which the syringe cone 42 is inserted. The withdrawal port 10 may be configured to retain in a clamped manner the syringe at the withdrawal port 10 without a user having to push the syringe further actively in the direction of the withdrawal port 10 after insertion in order to prevent the syringe from accidentally detaching from the withdrawal port 10.

The withdrawal port 10 comprises a closure section which, when using the withdrawal port 10, cooperates with the syringe cone 42 in order to automatically interrupt the liquid connection 15 between the orifices 22, 23 of the tube connections 12, 13 and simultaneously guarantee a fluid communication, in particular a liquid communication, between the syringe cone 42 and the first tube connection 12 when the syringe cone 42 is being inserted into the withdrawal port 10. The closure section is formed by a sealing surface 19 which is provided at a tapered housing section. When the syringe cone 42 comes into contact with the sealing surface 19, as shown in FIG. 3 and FIG. 4, the liquid connection 15 is interrupted. At the same time, the syringe cone 42 may communicate with the first tube connection 12 through a part 18 of the liquid connection 15 such that a liquid may flow between the first tube connection 12 and the syringe cone. Via the first tube connection 12, blood may be withdrawn from the first arm 2 of the tubing.

The aperture of the withdrawal port 10 and/or guide elements, such as ridges or other protrusions, provided at the aperture define an insertion direction 26 along which the syringe cone 42 can be inserted into the withdrawal port 10. The first tube connection 12 and the second tube connection 13 are configured such that they are offset from one another along the insertion direction 26. A first longitudinal axis 24 of the first tube connection 12 and a second longitudinal axis 25 of the second tube connection 13 may be offset from one another along the insertion direction 26. The first longitudinal axis 24 of the first tube connection 12 and the second longitudinal axis 25 of the second tube connection 13 may run parallel to each other. The housing 11 may form a shoulder 16 at which an inner cross-section of the housing tapers in order to guarantee that the sealing surface 19 abuts an end region 43 of the syringe cone 42 closely and in a substantially liquid-tight manner. The syringe cone 42 may be retained frictionally at its end section 43 within the housing, in particular clamped by the sealing section 19. Alternatively or additionally, the syringe cone 42 may be clamped by means of the seal 28 and/or the aperture in the housing 11, via which the syringe cone 42 is inserted.

Since the first tube connection 12 and the second tube connection 13 are arranged offset from one another, in the withdrawal port 10 the housing 11 redirects the liquid flow between the first tube connection 12 and the second tube connection 13 when the syringe cone 42 is not inserted into the withdrawal port 10 so far that a substantially tight contact with the sealing surface 19 has been established. If the syringe cone 42 is pushed through the seal 28, the seal 28 partly pushes sideways, partly may fold itself in into the housing 11, as is shown in FIG. 4. As soon as the syringe cone 42 has been inserted as far as it will go, the tip of the syringe cone 42 extends into the liquid connection 15 such that the liquid connection 15 is interrupted. As a reaction to the insertion of the syringe cone 42, the liquid connection between the first and second tube connections 12, 13 is thus automatically interrupted, and an aspiration of a liquid into the syringe may only take place by means of the first tube connection 12. It is not possible to draw in a mixture of blood and an infusion solution or another liquid via the second tube connection 13.

The withdrawal port 10 is configured such that the flow cross-section between the first tube connection 12 and the aperture of the syringe is not limited or reduced when the syringe cone 42 is inserted into the withdrawal port 10. As illustrated in FIG. 3 and FIG. 4, the flow path between the first tube connection and the receiving section 17 is not reduced during insertion of the syringe cone 42. Furthermore, neither the seal 28 nor another element of the withdrawal port 10 overlaps the aperture at the end of the syringe cone 42 when the syringe cone 42 is completely inserted. In particular, the seal 28 may be configured such that it abuts only the conical outer surface of the syringe cone 42, but simultaneously remains spaced apart from the axial end region of the syringe cone 42, and does particularly not even partially overlap the aperture at the end of the syringe cone 42 when the syringe cone 42 is completely inserted.

When the syringe cone 42 is removed again, the liquid connection 15 will automatically be opened again. Consequently, the user does not have to take additional steps in order to open and close the liquid connection 15 by means of separately manually actuating a three-way stopcock.

The withdrawal port 10 may be configured such that after blood withdrawal from the tubing, the flow path between the first tube connection 12 and the second tube connection 13 flushes itself again. For this purpose, a nose may be provided in the inlet facing away from the patient. The withdrawal port may be configured such that within the withdrawal port 10 there are no volumes at which the flow speed is zero when a liquid flows between the first tube connection 12 and the second tube connection 13.

The withdrawal port 10 may comprise a retaining plate 20 for particularly easy handling. The retaining plate 20 allows the withdrawal port 10 to be retained, e.g., between two fingers while the syringe 41 is inserted for blood withdrawal purposes.

FIGS. 5 to 8 show a system 1 with a withdrawal port 50 according to a further embodiment. Elements which, according to their configurations or functions, correspond to elements which have already been described with reference to FIGS. 1 to 4 are designated by the same reference signs.

FIG. 5 shows the withdrawal port 50 in a sectional view, wherein the section plane runs parallel to longitudinal axes 24, 25 of tube connections 12, 13, and the system 1 is illustrated in a state in which a liquid connection 15 is open. FIG. 6 shows the withdrawal port 50 in a sectional view, wherein the section plane runs perpendicular to the longitudinal axes 24, 25 of the tube connections 12, 13, and the system 1 is illustrated in a state in which the liquid connection 15 is open. FIG. 7 and FIG. 8 show the withdrawal port 50 in a view corresponding to the sectional views of FIG. 5 and FIG. 6 when the liquid connection 15 is closed. As will be described in more detail, the withdrawal port 50 comprises a closure section 53 which, when the syringe cone 42 is being inserted into the withdrawal port 50, is deformed such that the closure section 53 closes the second tube connection 13.

The withdrawal port 50 comprises a housing 11. The housing 11 may have a multipart design. For instance, the housing 11 may comprise two housing parts 11A, 11B, wherein each of the tube connections 12, 13 may be arranged at each of the housing parts 11A, 11B, respectively. The first and second tube connections 12, 13 may, e.g., be arranged coaxially, with the first longitudinal axis 24 of the first tube connection 2 and the second longitudinal axis 25 of the second tube connection 3 extending along the same straight line.

The housing 11 comprises an aperture 52 through which the syringe cone 42 can be inserted. The aperture 52 may define an insertion direction 26 of the syringe cone 42, optionally in combination with ridges or other guide elements at the aperture 52. The housing 11 may comprise a wall section 51 which tapers in the direction towards the second tube connection 13. In any case, in the tapered wall section 51 a closure section 53 may be provided within the housing 11. The closure section 53 may comprise or consist of an elastomer, e.g., silicone, latex, TPE, and/or EPDM. The closure section 53 may preferably comprise a protrusion 54 which is receivable in the aperture 52 to close and/or seal the aperture 52 when the syringe cone 42 is not inserted into the withdrawal port 50. In this context, an outer surface of the protrusion 54, which extends into the aperture 52 in the manner of a nose, may be substantially flush with an outer surface of the housing 11.

The protrusion 54 may be dimensioned such that it is flush with the region of the housing 11 surrounding the aperture when the syringe cone 42 is not inserted into the withdrawal port. The protrusion 54 may be dimensioned such that together with the housing 11 it provides a surface around the aperture of the housing 11 that is substantially free of steps. In doing so, wipe disinfection is facilitated. The protrusion 54 may be dimensioned such that it completely occupies the aperture of the housing 11 and forms an outer surface of the withdrawal port 50 that is free of steps when the syringe cone 42 is not inserted into the withdrawal port 50.

The housing 11 or a retainer optionally provided at the housing 11 may be configured to retain in a clamped manner the syringe cone 42 at a section of the syringe cone 42 that is spaced apart from an end region 43 of the syringe cone. The housing 11 or the retainer may particularly be configured to retain in a clamped manner the syringe cone 42 at the proximal end of the syringe cone 42. In doing so, an inwardly-facing clamping force may be exerted on the syringe cone 42. The clamping force may be directed substantially radially to the center axis of the syringe cone 42.

The withdrawal port 50 may thus be configured to retain in a clamped manner the syringe cone 42 at the withdrawal port 50 when the syringe cone 42 is inserted into the withdrawal port 50. The withdrawal port 50 may be configured such that the syringe cone 42 is retained in a clamped manner by the withdrawal port 50 without having to be threadingly engaged with the withdrawal port. The withdrawal port 50 may have a non-threaded outer surface. Disinfection of the outer surfaces is thus facilitated in the vicinity of the aperture through which the syringe cone 42 is inserted. The withdrawal port 50 may be configured to retain in a clamped manner the syringe at the withdrawal port 50 without a user having to push the syringe further actively in the direction of the withdrawal port 50 after insertion in order to prevent the syringe from accidentally detaching from the withdrawal port 50.

As can best be seen in the sectional view of FIG. 6, the closure section 53 may annularly surround the longitudinal axis 25 of the second tube connection 13 when the syringe cone 42 is not inserted into the withdrawal port 50. The insertion of the syringe cone 42 as far as it will go elastically and reversibly deforms the closure section 53 such that the closure section 53 deformed by the syringe cone 42 closes the second tube connection 13. A liquid communication between the syringe cone 42 and the first tube connection 12 remains possible.

The insertion direction 26 along which the syringe cone 42 can be inserted into the withdrawal port 50 may be inclined relative to the longitudinal axes of the first tube connection 12 and the second tube connection 13. In this context, the insertion direction 26 may form an obtuse angle with the longitudinal axis 24 of the first tube connection 12 in order to guarantee safe liquid communication between the syringe cone 42 and the first tube connection 12 even when the closure section 53 is elastically deformed by the syringe cone 42. The insertion direction 26 may form an acute angle, e.g., of at least 60°, with the longitudinal axis 25 of the second tube connection 12 in order to guarantee safe liquid communication between the syringe cone 42 and the first tube connection 12 even when the closure section 53 is elastically deformed by the syringe cone 42.

The closure section 53 may be configured to close the second tube connection 13 when the syringe cone 42 is inserted, and/or to close the aperture 52 of the housing 11 when the syringe cone 42 is not inserted. Optionally, the closure section 53 may also retain in a clamped manner the syringe cone 42 within the housing alone or in cooperation with the housing 11. For instance, a concave region 56 of the closure section 53 may be positioned such that an edge of the syringe cone 42 is at least partially engaged around and retained in a clamped manner by the closure section 53 deformed by the syringe cone.

The withdrawal port 50 is configured such that the flow cross-section between the first tube connection 12 and the aperture of the syringe is not substantially limited or reduced when the syringe cone 42 is inserted into the withdrawal port 50. As illustrated in FIG. 7, the flow path between the first tube connection and the receiving section 17 is not reduced upon insertion of the syringe cone 42. The closure section 53 may be configured such that it abuts only the conical outer surface of the syringe cone 42 but at best slightly or preferably not at all overlaps the aperture at the end of the syringe cone 42.

When the syringe cone 42 is removed from the withdrawal port 50, due to its elasticity the closure section 53 returns to the configuration shown in FIG. 5 and FIG. 6. The liquid connection 15 between the first tube connection 12 and the second tube connection 13 is opened again automatically.

The withdrawal port 50 may be configured such that after blood withdrawal from the tubing, the liquid path between the first tube connection 12 and the second tube connection 13 flushes itself again. The withdrawal port may be configured such that within the withdrawal port 50 there are no volumes at which the flow speed is zero when a liquid flows between the first tube connection 12 and the second tube connection 13.

With the withdrawal ports according to all embodiments, it is not imperative for the second tube connection to be closed in a completely fluid-tight manner. A reliable aspiration of blood through the first tube connection is achieved already when a flow resistance for a liquid between the first and the second tube connections is considerably greater than a flow resistance for a liquid between the syringe cone and the first tube connection and the vascular cannula when the syringe cone is inserted into the withdrawal port as far as it will go.

Withdrawal ports according to all embodiments may, e.g., be configured such that the first tube connection and the second tube connection are each connectable to arms of a tubing, which are conventional in medical applications. The first tube connection and the second tube connection may, e.g., each be configured so as to be connected with conduits having an outer diameter from 2 mm up to and including 4.5 mm, but also with conduits of other dimensions.

The withdrawal port according to different exemplary embodiments allows for the syringe to be retained in a clamped manner on the withdrawal port without having to use a Luer lock connection. This allows for the withdrawal port to be used with a plurality of syringes, in particular also with syringes which do not comprise a Luer lock connection.

The withdrawal port according to different exemplary embodiments forms an outer surface that is free of steps at the aperture through which the syringe cone is being inserted into the withdrawal port. In contrast to a withdrawal port which permanently comprises a recess at the aperture, disinfection of the withdrawal port is thus facilitated. In particular, wipe disinfection may be carried out in a simple fashion.

The withdrawal port according to different exemplary embodiments allows blood to be aspirated into the syringe without narrowing the flow cross-section. This way enables an efficient blood withdrawal which reduces the risk of damaging red blood cells due to occurring negative pressures.

While exemplary embodiments have been described with reference to the figures, numerous modifications may be implemented in further exemplary embodiments. For instance, closure sections which reversibly releasably come into contact with a syringe cone and, as a reaction thereto, interrupt the liquid connection between the first and the second tube connection may have other configurations than those described in detail.

With the withdrawal port according to the invention, the risk associated with incorrect operation of conventional three-way stopcocks is substantially reduced.

1. A withdrawal port for withdrawing a liquid, in particular blood, from a tubing, comprising:
a first tube connection which is connected or connectable with an arm of a tubing;
a second tube connection;
a fluid connection, in particular a liquid connection, between the first and second tube connections; and
a receiving section which is suitable for reversibly releasably receiving a syringe section, particularly a syringe cone, of a syringe,
wherein the withdrawal port is configured such that an insertion of the syringe section into the receiving section interrupts the fluid connection between the first tube connection and the second tube connection and causes fluid communication of the syringe section with the first tube connection.

2. The withdrawal port according to claim 1, further comprising:
a housing; and
a closure section which is formed or arranged on the housing, wherein the closure section is configured so as to reversibly releasably engage the syringe section in order to interrupt the fluid connection between the first tube connection and the second tube connection.

3. The withdrawal port according to claim 2,
wherein the housing comprises an aperture through which the syringe section is insertable into the receiving section of the withdrawal port, and
wherein the withdrawal port comprises a seal arranged at the aperture.

4. The withdrawal port according to claim 3,
wherein the seal comprises a protrusion which extends into the aperture such that an outer surface of the seal is substantially flush with the housing or with a retainer of the seal when the syringe section is not inserted into the housing.

5. The withdrawal port according to claim 4,
wherein the outer surface of the seal, which is substantially flush with the housing or the retainer, forms an outer surface of the withdrawal port without pronounced steps for wipe disinfection.

6. The withdrawal port according to claim 3,
wherein the seal and/or the housing is elastically deformable at the aperture for a sealing engagement with syringe cones of different diameters.

7. The withdrawal port according to claim 6,
wherein the seal comprises a nose for sealingly abutting a surface of the housing.

8. The withdrawal port according to claim 6,
wherein the housing and/or the seal is configured so as to frictionally retain, particularly in a clamped manner, the syringe section at a section of the syringe cone spaced apart from an end of the syringe cone.

9. The withdrawal port according to claim 3, further comprising
a retainer separate from the housing for retaining the seal.

10. The withdrawal port according to claim 9,
wherein the retainer is configured to retain in a clamped manner a syringe cone inserted into the receiving section.

11. The withdrawal port according to claim 9,
wherein the retainer comprises an annular shoulder partially engaging over the seal, said annular shoulder being configured to retain in a clamped manner the syringe cone inserted into the receiving section.

12. The withdrawal port according to claim 2,
wherein the closure section comprises a sealing surface formed on the housing of the withdrawal port for closing the second tube connection by way of contact of the syringe section with the sealing surface.

13. The withdrawal port according to claim 12,
wherein the first tube connection comprises a first longitudinal axis, and the second tube connection comprises a second longitudinal axis offset from the first longitudinal axis.

14. The withdrawal port according to claim 13,
wherein the housing is configured to redirect a liquid flow between the first tube connection and the second tube connection in the region of the receiving section and/or in the receiving section when the syringe cone is not inserted into the receiving section.

15. The withdrawal port according to claim 2,
wherein the closure section is elastically deformable in order to close the second tube connection during insertion of the syringe section into the receiving section.

16. The withdrawal port according to claim 15,
wherein, by means of inserting the syringe section into the receiving section, the elastically deformable closure section is reversibly convertible from a first configuration in which the closure section leaves open both the first tube connection and the second tube connection into a second configuration different from the first configuration in which the elastically deformable closure section closes the second tube connection.

17. The withdrawal port according to claim 16,
wherein the housing comprises an aperture through which the syringe section is insertable into the receiving section of the withdrawal port,
wherein the elastically deformable closure section is configured to close and/or seal the aperture in the first configuration.

18. The withdrawal port according to claim 15,
wherein the elastically deformable closure section consists of an elastomeric material.

19. The withdrawal port according to claim 2,
wherein the closure section is configured so as to frictionally, in particular in a clamped manner, retain the syringe section inserted into the receiving section.

20. The withdrawal port according to claim 2,
wherein the withdrawal port is configured so as to frictionally, in particular in a clamped manner, retain the syringe section at at least an end region of the syringe section within the housing.

21. The withdrawal port according to claim 2,
wherein the housing comprises protrusions, particularly ridges, for guiding or frictionally retaining the syringe section.

22. The withdrawal port according to claim 1, further comprising:
at least one retaining plate for retaining and/or fastening the withdrawal port on a patient's skin or on another structure.

23. The withdrawal port according to claim 1,
wherein a flow cross-section of a fluid connection between the first tube connection and the receiving section remains unchanged when the syringe section is being inserted into the receiving section.

24. The withdrawal port according to claim 1,
wherein the withdrawal port is configured so as to be automatically flushed after the syringe section has been removed from the receiving section.

25. A system, comprising:
a withdrawal port according to claim 1; and
a syringe with a syringe cone suitable for being reversibly releasably inserted into the receiving section of the withdrawal port in order to withdraw blood from a tubing by means of the withdrawal port.

26. The system according to claim 25,
wherein, by means of inserting the syringe cone into the receiving section, the system is convertible between a first configuration in which the syringe cone is arranged outside of the receiving section and the fluid connection between the first tube connection and the second tube connection is open and a second configuration in which the syringe cone is inserted into the receiving section of the withdrawal port, the fluid connection between the first tube connection and the second tube connection is interrupted, and the syringe cone is in communication with the first tube connection.

27. A use of the withdrawal port of claim 1 for withdrawing blood from an arm of a tubing.
